(19) United States
(12) Patent Application Publication
(10) Pub. No.: US 2024/0042110 A1
(43) Pub. Date: Feb. 8, 2024

(54) Vessel Analysis-Based Medical System for Specifying Adjustable Values of a Blood Treatment Apparatus

(71) Applicant: Fresenius Medical Care Deutschland GmbH, Bad Homburg (DE)

(72) Inventor: Florian Hubert, Bad Homburg (DE)

(21) Appl. No.: 18/264,295

(22) PCT Filed: Feb. 10, 2022
(86) PCT No.: PCT/EP2022/053219

§ 371 Date: Aug. 4, 2023

(30) Foreign Application Priority Data
Feb. 11, 2021 (DE) 10 2021 103 209.1

(51) Int. Cl. A61M 1/16; G16H 20/40; A61B 5/00; A61B 3/12

(52) U.S. Cl. CPC A61M 1/1605; G16H 20/40; A61B 5/4836; A61B 3/12; A61M 2202/0014; A61M 2202/0042; A61M 2202/0413; A61M 2205/126; A61M 2205/103; A61M 2205/127; A61M 2205/35; A61M 2205/502; A61M 2205/3327; A61M 2230/005

(57) ABSTRACT

The present disclosure relates to a medical system for specifying adjustable setting of a blood treatment apparatus having a calculation device with an input interface for entering and/or reading in the results determined by a diagnostic device for executing a retinal vessel analysis of a patent, respectively; an output interface for outputting at least one technical parameter value for the treatment of the patient and/or outputting at least one target treatment parameter value for a treatment of the patent using the blood treatment apparatus, and/or for outputting suggested changes of existing pre-set or pre-settings for the technical parameter value and/or for the target treatment parameter value.

### CROSS-REFERENCE TO RELATED APPLICATIONS

The present application is the national stage entry of International Application No. PCT/EP2022/053219, filed on Feb. 10, 2022, and claims priority to Application No. DE 102021103209.1, filed in the Federal Republic of Germany on Feb. 11, 2021, the disclosures of which are expressly incorporated herein in their entirety by reference thereto.

### TECHNICAL FIELD

The present disclosure relates to a medical system as described herein, to a blood treatment apparatus as described herein, to a treatment system as described herein, to a method as described herein, to a digital storage medium as described herein, to a computer program product as described herein, and to a computer program as described herein.

### BACKGROUND

For the purpose of blood treatment, adjustable values for variable treatment parameters must be entered by a user, e.g., the clinic staff, or existing pre-set specifications must be adjusted, i.e., changed, on the blood treatment apparatus used for this purpose. The user of the blood treatment apparatus may conduct or initiate the adjusting or change of such adjustable values, usually manually, for example by input interfaces, e.g., on a screen (touchscreen) or on other interfaces suitable for input.

In order to achieve predetermined treatment targets or pre-settings, such as the renal dose or the ultrafiltration volume during dialysis treatment, it may be necessary for the user, in order to optimize the course of the treatment session, to take into account a number of individual circumstances and conditions of the patient when defining the adjustable values, which are here worded as technical parameter values (such as pumping rates of the involved pumps such as the blood pump or the ultrafiltration pump) or as target treatment parameter values (such as the renal dose or the duration of the treatment session, which are formulated as goals, but at the same time are implemented or pursued by setting technical parameters). The individual circumstances and conditions of the patient may include the patient's physical condition or vital signs

An aspect of the present disclosure includes a description of a medical system for assisting the user in establishing adjustable values for the treatment by a blood treatment apparatus, as well as a blood treatment apparatus comprising a medical system, a treatment system, and a method. Furthermore, a digital storage medium, a computer program product, and a computer program are described herein.

### SUMMARY

A medical system (hereinafter also shortly: system) is described by the present disclosure for specifying adjustable values (or ranges of values therefor) of a blood treatment apparatus, or for assisting the user of the blood treatment apparatus thereby, i.e., for specifying values (or ranges therefor) which are variable and could be set, for example, by the physician at the blood treatment apparatus before or during the treatment session at, or with an effect on, the blood treatment apparatus.

The medical system described herein comprises a calculation device with an input interface. The latter is prepared for a manual input or for the automatic reading in of results that have been determined by a diagnostic device (also: examination device), which is designed to carry out a retinal vessel analysis of a patient, beforehand or previously and are therefore already available.

The system further comprises an output interface. It is configured to output at least one technical parameter value, alternatively or additionally at least one target treatment parameter value, and/or changes from existing pre-set specification or pre-settings for the technical parameter value and/or for the target treatment parameter value. Technical parameter value and target treatment parameter value each serve, directly or indirectly, to control or regulate the blood treatment apparatus during a treatment session of the patient using same.

In this, the calculation device is programmed, based on the results for the technical parameter value and/or the target treatment parameter value or their levels or amounts, entered or read in by the first input interface, to determine or assign for each of them numerical values, or amounts for controlling or regulating the blood treatment apparatus for treatment of the patient.

A determining or specifying of data may be or may encompass examining of an existence or non-existence, measuring, inferring, calculating, obtaining, attaining, and/or recognizing.

Alternatively or additionally, the calculation device is programmed to determine or assign, based on the results entered or read in by the first input interface, any required changes (or their level, value, amount, etc.) with respect to a previous specification of the technical parameter value or of the target treatment parameter value.

The previous specification may be, for example, a pre-set specification already set, for example, on the blood treatment apparatus, or a pre-setting, for example, a value previously used for treatment, an entry in the patient's record or history, an instruction by the attending physician, etc.

The calculation device is further programmed to output, via the output interface, the technical parameter value and/or the target treatment parameter value, respectively determined by it and/or to output the respective change determined by it.

The blood treatment apparatus described herein, which is preferably designed as a dialysis apparatus, comprises a medical system as described herein or is connected thereto in signal connection or signal communication.

The treatment system described herein comprises, or consists of, one or several blood treatment apparatuses, preferably as disclosed herein, and a medical system as described herein.

In this, at least one blood treatment apparatus is preferably configured as a dialysis apparatus.

One, several or all of the blood treatment apparatus(es) of the treatment system on the one hand and the medical system on the other hand are present separately from each other.

In this, the term “separate from each other” may include, for example, a spatial, physical separation and/or a separation such that there is no signal communication between the blood treatment apparatus and the medical system. Alternatively or additionally, the term “separate from each other” may be understood to mean that one component (e.g., the medical system) is not part of the other component (e.g., one of the blood treatment apparatuses).

The method described herein preferably serves to prepare an upcoming treatment or treatment session of a patient, which is to be carried out by using a blood treatment apparatus, and the method preferably ends already before the start of the treatment. Alternatively or in addition, the method described herein runs or goes on during the treatment session by the blood treatment apparatus.

The method encompasses:
providing a medical system as described herein, a blood treatment apparatus, a treatment system; alternatively, said method encompasses using such a medical system, blood treatment apparatus or treatment system being already provided;
manually entering at least one result which was captured by the diagnostic device into the input interface of the calculation device;
reading of at least one technical parameter value and/or at least one target treatment parameter value, or the respectively suggested changes therefor being outputted by or at the output interface for a treatment of the patient by using the blood treatment apparatus; and
entering the read technical parameter value and/or target treatment parameter value, or their respective change, as an adjustable value into an input interface of the blood treatment apparatus.

A digital, particularly non-volatile storage medium, particularly in the form of a machine readable carrier, particularly in the form of a diskette, CD, DVD EPROM, FRAM (Ferroelectric RAM), NOVRAM or SSD (Solid-State-Drive), particularly with electronically or optically readable control signals, is designed such that it interacts with a programmable computer system, so that a conventional calculation device is reprogrammed into a calculation device of a medical system as described herein, e.g., when its storage content is running on the programmable computer system.

A computer program product described herein comprises a volatile, transient program code or one being saved on a machine-readable carrier, or a signal wave, configured to interact with a programmable computer configuration of a computer system such that a conventional calculation device is reprogrammed into a calculation device of a medical system as described herein.

A computer program product may be understood according to the present disclosure as a computer program stored, e.g., on a medium, an embedded system being a comprehensive system with a computer program (e.g., electronic device with a computer program), a network of computer implemented computer programs (e.g., client/server-system, a cloud computing system etc.), or a computer on which a computer program is loaded, runs, is stored, is executed or developed.

The term “machine readable carrier” as it is used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

According to the present disclosure, a computer program product may also be understood to mean a programmed application (in short: app), for example, particularly for a smartphone, a tablet or for any other mobile handheld device.

A computer program described herein comprises a program code, in order to effect that a conventional calculation device can be reprogrammed to be a calculation device of a medical system as described herein if the computer program is running on a corresponding computer system. According to the present disclosure, a computer program can, for example be taken to mean a physical, distributable software-product, which comprises a program.

In all of the aforementioned or the following embodiments, the use of the expression “may be” or “may have” and so on, is to be understood synonymously with the expression “preferably is” or “preferably has,” and so on respectively, and is intended to illustrate embodiments.

Embodiments according to the present disclosure may comprise one or several of the aforementioned or following features. In this, the features mentioned herein may, in any combination, be subject-matter of embodiments according to the present disclosure, unless the person skilled in the art recognizes a specific combination as technically impossible.

Whenever numerical words are mentioned herein, the person skilled in the art shall recognize or understand them as indications of numerical lower limits. Unless it leads the person skilled in the art to an evident contradiction, the person skilled in the art shall comprehend the specification for example of “one” as encompassing “at least one”. This understanding is also equally encompassed by the present disclosure as the interpretation that a numeric word, for example, “one” may alternatively mean “exactly one”, wherever this is evidently technically possible for the person skilled in the art. Both understandings are encompassed by the present disclosure and apply herein to all used numerical words.

Whenever a suitability or a method step is mentioned herein, the present disclosure preferably also encompasses the corresponding programming or configuration of a suitable apparatus, in particular an apparatus as described herein, or of a section thereof.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer. Therefore, for every embodiment mentioned herein it applies that the converse embodiment, e.g., formulated as negation, is also disclosed.

When an embodiment is mentioned herein, it is then an exemplary embodiment according to the present disclosure.

Whenever “programmed” or “configured” is mentioned herein, it is also disclosed that these terms can be interchanged with each other.

If “UF” is used herein, it is then a common abbreviation for ultrafiltration.

If “DVA” is used herein, it is then a common abbreviation for dynamic retinal vessel analysis.

In the retinal vessel analysis, a retinal camera (also: fundus camera), the Retinal Vessel Analyzer (see, for example, DE 10 2004 017 130 B4, DE 10 2006 018 445 A1, DE 196 48 935 B4 and others), measures the diameter of arteries and veins of the eyeground.

In dynamic retinal vessel analysis (“DVA”), additional stimulation is applied with a 12.5 Hz optoelectric flicker light and the change in vessel diameter is quantified. This test may follow different protocols, for example, three times 20 seconds of flicker light with a wavelength, for example, between 510 nm and 580 nm with, for example, 80 seconds of recovery time in between. The examination is usually performed with the patient's pupils dilated with medication; it is completely non-invasive and can provide information about a patient's cardiovascular risk profile.

In several embodiments, the results from the retinal vessel analysis determined in advance using the diagnostic device particularly encompass, or build upon, venous and/or arterial dilatation.

If, as provided in several embodiments, the maximum diameter of the vessel of the eyeground under consideration or the retinal diameter is divided by its basic or base diameter (also known as “initial value”, “resting diameter” or “baseline”, see also the following mentioned dissertation by Karoline Bornmann on this subject), a percentage value is achieved which may serve as a result.

In certain embodiments, the determined values of venous and/or arterial dilatation may be displayed as a graph. Thus, a long-term course can be presented to the attending physician. Based on increasing values of venous and/or arterial dilatation a cardioprotective dialysis treatment may be indicated.

In some embodiments, the system comprises a display device.

The display device is configured or programmed to display the results from the examination performed by the diagnostic device. Alternatively or additionally, the display device is programmed to display the technical parameter value and/or target treatment parameter value determined by the calculation device of the system, or to display their respective, optionally suggested, change.

In several embodiments, the results are or have been assigned to percentiles, tertiles, further classification units, etc., i.e., related to the results from measurements obtained from a collective of patients. Each percentile, tertile, further classification unit, etc. may be associated with a value or range of values for one or more technical parameters and/or for one or more target treatment parameters, e.g., in data storages, reference tables, etc.

In such data storages, reference tables, etc., there may be stored values or ranges of values for one or more technical parameters and/or for one or more target treatment parameters, which have been specified or determined by (or from) models, algorithms, artificial neural networks, artificial intelligence, comparison with a collective of patients and, e.g., their respective results and/or courses retinal vessel analyses and/or in particular using machine learning.

In such data storages, reference tables, etc., which may be part of the system or treatment system described herein, data may in several embodiments be modifiable or, e.g., based on one's own experience or after learning of current research findings, adaptable by some individuals, e.g., medical personnel, technicians, or others, with or without prior verification of eligibility or authorization (such as requesting a password, biometric data, etc.).

Thus, the medical system or treatment system may encompass an interface for modifying data stored in data storages, reference tables, etc.

In some embodiments, the output interface is, or comprises, a visual or graphic interface for the user. Alternatively, it is connected to a graphical display device, for example a monitor or a printer.

In several embodiments, the output interface may be designed as a graphical user interface (GUI) and/or may be a user interface herein.

In some embodiments, the display device is programmed or configured to display values of the technical parameter or of the target treatment parameter as described herein.

In several embodiments, the output interface is, or encompasses, an interface with/to a control device or closed-loop control device of a blood treatment apparatus.

In some embodiments, the output interface is, or encompasses, an interface with a server, a network protocol, and/or a data storage.

In several embodiments disclosed herein, the medical system is, or comprises, a mobile device, handheld device, cell phone, smartphone, tablet, etc.

Alternatively or in addition, the system is, or comprises, an application being suitable for this purpose.

The application may be downloaded or stored on a mobile device or on another device, or said application may be executed by, or may run on, a mobile device or another device.

In some embodiments, the medical system further comprises a diagnostic device or examination device for performing a retinal vessel analysis of a patient. This can be designed, for example, as described in one of the aforementioned property rights or property right applications. Their respective disclosure is therefore herein made by reference the subject-matter of the present disclosure as well.

The diagnostic device may be designed as described in the medical dissertation of Karoline Bornmann (“Influence of flicker light on retinal vessel diameter taking into account the female hormonal cycle within one month” from 2014, submitted to the University of Jena). Its related disclosure content is made by reference the subject matter of the present disclosure as well.

Retinal vessel analysis may be static or dynamic, i.e., using flicker light, as set forth at the outset.

In several embodiments of the medical system, the technical parameter values or target treatment parameter values determined by the computing device relate to or encompass
the type of treatment and/or
the ultrafiltration volume and/or
the treatment duration and/or
the sodium management (e.g., the amount of sodium which as a target value should be withdrawn by dialysis during the treatment session or the concentration of sodium in the patient's blood at the end of the treatment session, for which it may, for example, be specified to correspond to that concentration at the beginning of the treatment session; it may, for example, correspond to 138 mmol/l) and/or
the liquid management, in particular the conveyance rate of the blood pump and/or the conveyance rate of the substitute fluid pump
   or, respectively, changes thereof. In this, these values relate to the current or upcoming treatment session of the patient to be treated.

A renal dose may be defined as a quotient of effluent flow or filtrate flow on the one hand and patient weight on the other.

When determining the effluent flow, different flows (e.g. dialysate flow, dialysis liquid flow, substitute fluid flow, calcium flow, citrate flow, net ultrafiltration) may have to be taken into account, depending on the type of dialysis treatment.

For example, the renal dose may be the target renal dose (TRD) or the reached renal dose (RRD).

The target renal dose (TRD) may be calculated, for example, using the following formula:

$$TRD = \frac{PostDF + DF + NFRF + \frac{PreDF}{1 + \frac{PreDF}{BF}}}{m}$$

In this regard, the following applies
BF blood flow or blood flow rate
DF dialysis fluid flow or dialysis fluid flow rate
NFRF net fluid removal flow or net fluid removal flow rate
m patient's weight/mass
PostDF post-dilution flow or post-dilution flow rate
PreDF pre-dilution flow or pre-dilution flow rate.

The reached renal dose (RRD) can be calculated or determined depending on the treatment duration t using the formula

$$RRD = \frac{\frac{PostDV}{t} + \frac{DV}{t} + \frac{NFRV}{t} + \frac{\frac{PreV}{t}}{1 + \frac{\frac{PreDV}{t}}{\frac{BV}{t}}}}{m}$$

wherein the following applies:
BV processed blood volume
DV dialysis fluid volume
NFRV net fluid removal volume
m patient's weight/mass
PostDV post-dilution volume
PreDV pre-dilution volume

In some embodiments, the medical system comprises, or is, a control device or closed-loop control device of a blood treatment device or is part thereof. Alternatively, the medical system is in signal connection or in signal communication with such a system.

When a signal connection or signal communication between two components is mentioned herein, this may be understood to mean a connection that is taking place during use. Likewise, it may be understood to mean that there is a preparation for such a signal communication (in a wired, wireless or in another manner) for example by coupling the two components, for example by pairing, etc.

Pairing is understood as a process that takes place in connection with computer networks to establish an initial link or connection between computer units for the purpose of communication. The best-known example of this is the establishing of a Bluetooth connection, by which various devices (e.g., smartphone, headphones) are connected with each other. Pairing is sometimes also referred to as bonding.

In several embodiments, the control device or closed-loop control device is programmed to control or regulate the blood treatment apparatus, which comprises a conveyance device for a liquid, in particular an ultrafiltration pump, a blood pump, and/or a dialysis fluid pump, based on the calculated or determined technical parameter value or target treatment parameter value.

In some embodiments, the blood treatment apparatus is designed as a hemodialysis apparatus, hemofiltration apparatus or hemodiafiltration apparatus, in particular as an apparatus for the acute renal replacement therapy, for the chronic renal replacement or for the continuous renal replacement therapy (CRRT).

When reference is made herein to a pre-set value, or a pre-setting, this may be a last set value, a standard value, a displayed value, or a default-setting. A value set ex-works or by the service technician is herein to be understood as being optional, but not as being mandatory.

In several embodiments, the input interface may optionally be used to optionally change the determined or proposed value of a parameter value and/or a target treatment parameter value by a user, such as by manual manipulations at the input interface or optionally by manipulating its switches, keys, etc.

In several embodiments, the calculation device is connected to, or prepared to be connected to, the output device and/or to the input interface, preferably in signal communication.

In some embodiments, the input interface may comprise or be a correspondingly designed touchscreen, a rotary switch, a slider, a keyboard or the like.

In certain embodiments, it may be provided to automatically read in results at the input interface as well as to allow manual input.

Taking into account the results determined by the diagnostic device when determining the target treatment parameter value (or its level) or the technical parameter value (or its level) or their respective change for components of the blood treatment apparatus, for example for components such as blood pump and/or dialysis fluid pump, may in several embodiments be done for example based on known algorithms, which in turn may include further parameter values for example from auxiliary tables, etc. These algorithms and/or further parameter values (as well as further adjustable values belonging to the latter) may for example be stored in a storage device of the medical system.

The technical parameter value or target treatment parameter value, in particular their change, may be actively confirmed or rejected by the user in some embodiments. Suitable input options for this, for example a save or cancel button, can be provided.

Confirming may be a simple pressing of an “OK” button or the like.

Confirming, in several embodiments, does not encompass typing, entering, selecting, etc., of adjustable values from a plurality of options, even less calculating them or tacitly “nodding them off” in passing or in one's head.

The results obtained by the diagnostic device, the technical parameter values, target treatment parameter values and/or treatment specification(s) preferably refer to the specific patient being treated at this moment by the blood treatment apparatus or to a specific patient whose blood treatment by the blood treatment apparatus is due in the near future.

In several embodiments, the technical parameter values determined by the calculation device are or encompass an adjustable value for the blood pump and an adjustable value for the dialysis fluid pump. In other words, the calculation device is in these embodiments programmed to display or differently output, using the display device, the adjustable value for the blood pump and the adjustable value for the dialysis liquid pump as information to the user as technical parameter values, or as a part thereof, wherein said adjustable values have been determined or calculated, respectively, based on the technical parameter value by taking into account the results from the examination performed by the diagnostic device.

According to the present disclosure, the medical system is in several embodiments implemented in a control device or closed-loop control device of a blood treatment apparatus, whereby the blood treatment apparatus is in turn configured to be a blood treatment apparatus as described herein. In these embodiments, already existing input and output interfaces as well as the calculation device of the blood treatment apparatus could then be advantageously used to implement the medical system described herein.

In some embodiments, the medical system may comprise or be a server-based solution in which the user may retrieve, for example via a website, a program running on the server to run or initiate the steps of the method described herein. Thus, the medical system may optionally comprise a server. It may comprise user interfaces and/or user terminals such as a computer, cell phone or tablet, which are set up to communicate with the server.

In several embodiments, the calculation device of the medical system is programmed in such that it is capable of acquiring further (relevant) parameter values for the treatment session using existing or further input interfaces. Alternatively, further parameters may be read in, for example from a storage medium provided for this purpose. Alternatively or additionally, in some embodiments, the medical system is programmed to display the further parameter values in existing or further output interfaces. Advantageously, the calculation device may be configured to take these parameter values into account when determining the adjustable values for the involved pumps of the blood treatment apparatus.

In some embodiments, the calculation device of the medical system is programmed to display the adjustable value for the blood pump and the adjustable value for the dialysis liquid pump of the selected combination, using the display device, in order to inform the user.

In several embodiments, the output interface and the display device may be identical. Thus, an output by the output interface may be a display by the display device, and vice versa.

The medical system may be programmed to transfer the values determined or assigned by the calculation device, for example, to a logistics system and/or a billing system. The logistics system and/or billing system, which may be provided external to the medical system described herein but may be part of the system described herein, may in turn be programmed to monitor, record, store, output, bill and/or differently process supply flows, orders, consumption and the like. For example, the logistics system may be configured to use values transmitted by the medical system to reorder liquids consumed during a treatment session, such as citrate solution, dialysis liquid, etc., and deliver them to the clinic where the blood treatment was performed. The billing system may be provided to use the values transmitted by the medical system to bill for services provided by the clinic in connection with the treatment session. For example, the changed values of the target treatment parameters or the input of a treatment specification may reflect the consumption of liquids such as heparin, citrate solution, etc. This may be made as the basis for billing to the patient or a payer such as an insurance company.

In some embodiments, the method comprises controlling or regulating, preferably automatically, the blood treatment apparatus based on, as described herein, modified or input values and technical parameter values and/or target treatment parameter values determined thereupon.

In several embodiments, the diagnostic device is provided as an independent device, e.g., in a dialysis ward.

In some embodiments, the diagnostic device is or may be connected to a network (for example, of the clinic), and the respective results are transmitted, when in use, to a server that stores the results and transmits them to the blood treatment apparatus(es). The attending physician can view the results and set or modify technical parameter values or target treatment parameter values based on these results and by using the medical system as disclosed herein.

In several embodiments, the diagnostic device is attached or mounted to a blood treatment apparatus, for example pivotally, e.g., by one or more ball joints. In the example of a device for the retinal vessel analysis as a diagnostic device, for example the device for the retinal vessel analysis may be fixedly mounted to the blood treatment apparatus, while the fundus camera is pivotally mounted by a joint. By using several ball joints, the camera may be optimally aligned with the patient's eyeground. After use, the camera can be swiveled back to the back of the blood treatment apparatus.

In some embodiments, it is provided that the diagnostic device is designed similarly to “VR” glasses. In this, the diagnostic device may be provided in a housing that may accommodate both the fundus camera and the evaluation electronics. This housing may be put on by the patient and worn “like a pair of glasses” in order for the patient to undergo a measurement. In several embodiments, the results obtained thereby are transmitted, by cable or wirelessly, to the medical system or to the blood treatment apparatus.

In some embodiments, the results obtained from the examination using the diagnostic device preferably have an influence on the ultrafiltration rate (in short: “UF rate”) as an example of a technical parameter. Here, for example, depending on the time that has already passed since the start of the treatment session (also: t₀), the standard curve profile of the UF rate is changed over time based on the results of the examination performed by the diagnostic device. In this, different curve shapes are possible, which is described in detail in connection with FIG. 3 to FIG. 5. In such a case, the adjustment of the technical parameters may serve to withdraw the ultrafiltration volume (alternatively: ultrafiltration amount) in the most cardioprotective way possible by initially setting the UF rate and modifying it, in the best possible way, over the time of the treatment session with regard to the results of the retinal vessel analysis.

In several embodiments, the results of the examination performed by the diagnostic device preferably influence the duration of a blood treatment, wherein the duration is an example of a value of a target treatment parameter.

With an unfavorable calcium/phosphate balance, calcification causes increased stenoses in the vascular system. The excess phosphate may be broken down more effectively by prolonging the blood treatment.

In some embodiments, the results of the examination performed by the diagnostic device preferably influence the treatment type (in particular High Volume HDF) as another example of a target treatment parameter. If this treatment type is supported by the blood treatment apparatus, then based on the aforementioned results, for example, a High Volume HDF treatment may be activated by entering or setting “High Volume HDF” as a target treatment parameter value or an adjustable setting using the input interface of the blood treatment apparatus.

In some embodiments, the results of the examination performed by the diagnostic device preferably influence the sodium management. If a sodium management with 0-balance, i.e., the patient's sodium level should be at the same value before and after treatment, is supported by the blood treatment apparatus, then a sodium management with 0-balance may be activated on the blood treatment apparatus based on the results of the diagnostic device.

In several embodiments, the output interface for outputting at least one technical parameter value for treating the patient and/or of at least one target treatment parameter value for treating the patient by using the blood treatment apparatus is additionally designed to output a treatment notification. This notification may indicate a cardiologic risk for the specific patient as a warning for the physician, a recommendation, e.g., a drug, a substance of a group of active agents such as, e.g., “prescribe/administer anti-inflammatory drug” or the like. The calculation device may be programmed to generate such notification and/or to determine them from storage devices with regard to the results of the examination performed by the diagnostic device.

Some or all of the embodiments according to the present disclosure may have one, several or all of the advantages mentioned above and/or below.

Overhydration is common in patients with kidney failure and is closely associated with cardiovascular disease, the leading cause of death in dialysis patients. Achieving optimal liquid balance is therefore one of the key challenges in the dialysis routine.

An advantage of the present methods, systems, and devices may be to enable or facilitate for the user, i.e., mostly the attending physician, to set optimal technical parameter values or target treatment parameter values on the blood treatment apparatus used for the upcoming or current treatment session and to increase patient safety.

Since retinal vessel analysis may reflect a non-invasive indication of a patient's cardiovascular risk profile and adjustments to blood treatment may be derived therefrom in accordance with the present disclosure, the present systems, methods, and devices advantageously enables appropriate adjustment of technical parameter values or target treatment parameter values in patients who require special protection with respect to their cardiovascular condition.

It is thus advantageously possible by the present systems, methods, and devices to initiate a cardioprotective blood treatment using the blood treatment apparatus, the treatment being individually adapted to the patient to be treated. This indirectly increases the patient's quality of life and also contributes to his safety.

A further advantage of the present disclosure may further be that facilitating the finding of suitable technical parameter values and target treatment parameter values by the present methods, systems, and devices may provide more safety, in particular also, to an inexperienced user when carrying out the setting.

The probability of error in finding such values may be significantly reduced according to the present disclosure, for example because mental arithmetic steps or the risk of an error in reasoning done by the user may be eliminated.

The competence and responsibility for finding or changing the adjustable values, may nevertheless advantageously continue to be up to the user. The user thus receives valuable support from the medical system described herein, however, without the possibility of being surprised by its unauthorized action or handling.

If the blood treatment apparatus and the medical system remain to be separate devices, existing blood treatment apparatuses may be combined with the medical system described herein to form the treatment system described herein. Since interventions in the control system of the blood treatment apparatus are not necessary in this case, there is advantageously no need for renewed approval of the blood treatment apparatus as a medical device.

### BRIEF DESCRIPTION OF THE DRAWINGS

In the following, the present disclosure is exemplarily described based on the accompanying drawing in which same reference numerals designate identical or similar components. The following applies in the figures:

FIG. 1 shows a highly simplified representation of a medical system in addition to a blood treatment apparatus;

FIG. 2 shows the treatment system described herein in a first embodiment;

FIG. 3 shows the possible specification of a technical parameter value (here the ultrafiltration rate) when using the present systems, methods, and devices over time;

FIG. 4 shows the dependence of the withdrawn ultrafiltration volume on venous dilatation in an embodiment; and

FIG. 5 shows the dependence of the duration of a treatment session on venous dilatation in an embodiment.

### DETAILED DESCRIPTION

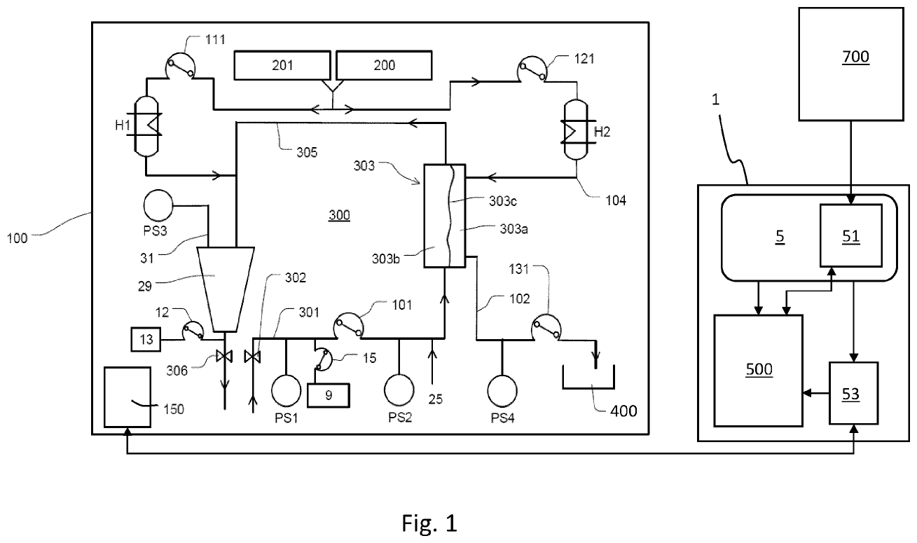

FIG. 1 shows, in a highly simplified representation, a medical system 1 in a first embodiment in addition to a blood treatment apparatus 100, which is optionally connected to an extracorporeal blood circuit 300. FIG. 1 thus shows a treatment system of an exemplary embodiment.

The extracorporeal blood circuit 300 comprises a first line 301, here in the form of an arterial line section.

The first line 301 is in fluid communication with a blood treatment device, here for example a blood filter or dialyzer 303. The blood filter 303 comprises a dialysis liquid chamber 303a and a blood chamber 303b, which are separated from each other by a mostly semi-permeable membrane 303c.

The extracorporeal blood circuit 300 further comprises at least one second line 305, here in the form of a venous line section. Both the first line 301 and the second line 305 may be used to connect them to the vascular system of the patient P, not shown.

The first line 301 is optionally connected to a (first) tube clamp 302 for blocking or closing the line 301. The second line 305 is optionally connected to a (second) tube clamp 306 for blocking or closing the line 305.

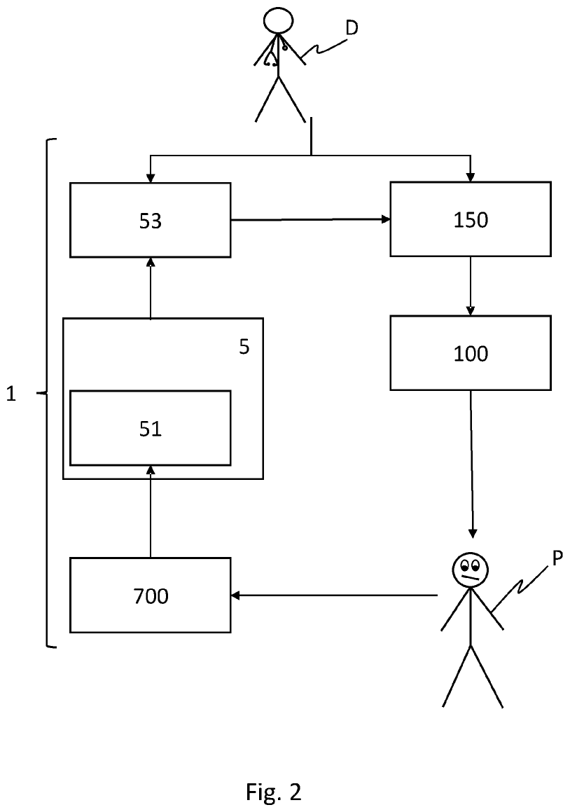

The blood treatment apparatus 100 represented in FIG. 1 schematically and only by some of its devices, comprises a blood pump 101. During the treatment of the patient P (see FIG. 2) the blood pump 101 conveys blood through sections of the extracorporeal blood circuit 300 and in the direction of the blood filter or dialyzer 303 as shown by the small arrows, which generally indicate in each of the figures the direction of flow.

Using a pump for dialysis liquid 121, that may be embodied as a roller pump or as any otherwise occluding pump, fresh dialysis liquid is pumped out of a source 200 along the dialysis liquid inlet line 104 into the dialysis liquid chamber 303a. The dialysis liquid leaves the dialysis liquid chamber 303a as dialysate, possibly enriched with filtrate, towards an optional effluent bag 400 and will be referred to herein as effluent.

The source 200 may for example be a bag or a container. The source 200 may further be a fluid line, for example, a hydraulic outlet or hydraulic port of the blood treatment apparatus 100, from which liquid is provided that is on-line and/or continuously generated or mixed.

A further source 201 with substitute fluid may optionally be provided. It may correspond to the source 200 or be a separate source.

An only outlined control device or closed-loop control device 150 can be configured to control or regulate the blood treatment session.

Where the effluent bag 400 is connected to the blood treatment apparatus 100 is indicated in the bottom right within the blood treatment apparatus 100 of FIG. 1.

In addition to the aforementioned blood pump 101 and the aforementioned pump 121 for dialysis liquid, the arrangement shown in FIG. 1 further comprises, purely optionally, a number of other optional pumps, namely the pump 111 for substitute fluid and the pump 131 for the effluent. The pump 131 may optionally be used in terms of ultrafiltration for establishing an underpressure

The pump 121 is provided to supply dialysis liquid to the blood filter 303, out of a source 200, for example a bag, through an optional available bag heater H2 having a heater bag, using the dialysis liquid inlet line 104.

The thus supplied dialysis liquid exits from the blood filter 303 via a dialysate outlet line 102 (also: effluent inlet line), supported by the optional pump 131, and may be discarded.

An optional arterial sensor PS1 is provided upstream of the blood pump 101. It measures the pressure in the arterial line during a treatment of the patient P (not shown here).

A further optional pressure sensor PS2 is provided downstream of the blood pump 101, but upstream of the blood filter 303 and if provided, upstream of an optional addition point 25° for Heparin. It measures the pressure upstream of the blood filter 303 (“pre-hemofilter”).

To measure the filtrate pressure of the blood filter 303 a further pressure sensor may be provided as PS4 downstream of the blood filter 303, however, preferably upstream of the pump 131 in the dialysate outlet line 102.

Blood, which leaves the blood filter 303, passes through an optional venous blood chamber 29, which may comprise a de-aeration device 31 and/or a further pressure sensor PS3.

The control device or closed-loop control device 150 shown in FIG. 1 may be in cable or wireless signal communication to any of the components referred to herein —especially or in particular to the blood pump 101—in order to control or regulate the blood treatment apparatus 100.

The optional pump 111 is provided to supply substitute fluid from the optional source 201, for example a bag, via an optional available bag heater H1 having a heater bag, to the second line 305.

In some embodiments, a citrate solution is delivered, if necessary by a citrate pump 15, into line 301 from an optionally provided source for citrate solution, designed here, e.g., as a citrate bag 9. For example, 4% Na₃ citrate is supplied from the source for citrate solution.

An optional addition device, designed here as a calcium pump 12, is provided in order to deliver a calcium solution into the line 305 from an optional source for calcium solution in FIG. 1, designed for example as a calcium bag 13. For example, a CaCl₂ solution is supplied from the source for calcium solution. This may have a calcium concentration of 91 mmol/l, 100 mmol/l or another suitable calcium concentration.

To the right of the blood treatment apparatus 100, FIG. 1 shows a medical system 1 with a calculation device 5 The calculation device 5 comprises an input interface 51 for manually inputting or automatically reading in results which have been determined by a diagnostic device 700 (also: examination device) for performing a retinal vessel analysis of a patient P (see FIG. 2).

The medical system 1 comprises an output interface 53 for outputting at least one technical parameter value for the blood treatment apparatus 100 for treating the patient P (not shown in FIG. 1) or at least one target treatment parameter value for a treatment of the patient P using the blood treatment apparatus 100.

The calculation device 5 is programmed to determine, based on results entered or read in by the input interface 51, the target treatment parameter value or its level or the technical parameter value or its level, or their respective suggested or advised change with respect to a pre-set specification on the blood treatment apparatus 100 or with respect to a specification for the control or regulation of the blood treatment apparatus 100 for the treatment of the patient P. In this, the change may be or may encompass, for example, a change with respect to a value previously used for treatment, an entry in the patient file or in patient history, etc.

Further, the calculation device 5 is programmed to output the determined technical parameter value and/or the determined target treatment parameter value, or their respective change for the operation of the blood treatment apparatus 100, by an output interface 53.

Such an output may be carried out for example at a display device 500. This may be configured or programmed to display the results of the examination being performed by or using the diagnostic device 700, the determined parameter values and/or the determined target treatment parameter values or their respective changes.

The display device 500 may optionally be a printer.

A storage device for storing or providing results of the examination being performed by the diagnostic device, or storing such results, is provided by the present disclosure in several embodiments.

Alternatively or additionally, the present system comprises at least one device which enables data (in particular results of the examination being performed by the diagnostic device, parameter values and/or target treatment parameter values) to be stored, provided and/or displayed over time (i.e., as a patient history). The storage, providing and/or display of data, for example as mentioned herein, relating to patient collectives, are also encompassed by the present disclosure.

Blood treatment apparatus 100 and medical system 1 together represent an embodiment of a treatment system.

The medical system 1 may be provided on a mobile handheld device, e.g., cell phone or tablet, spatially separated from the blood treatment apparatus 100.

In certain embodiments, the medical system 1 and the blood treatment apparatus 100 are separate from each other. The term “separate from each other” may encompass herein, for example, a spatial separation, a physical separation, and/or a separation such that there is no signal communication between the blood treatment apparatus 100 and the medical system 1. Alternatively or additionally, this may be understood to mean that one component (e.g., the medical system 1) is not part of the other component (e.g., the blood treatment apparatus 100).

In the embodiment shown in FIG. 1, the output interface 53 of the medical system 1 comprises an interface with the control device or closed-loop control device 150 of the blood treatment apparatus 100 or is connected to it in signal communication and/or physically or is part thereof.

In the latter cases, however, the devices thus connected are no longer a treatment system, but a blood treatment apparatus 100.

The medical system 1 may be or may comprise a server-based solution, i.e., comprise an interface to a server, a network protocol, and/or a data storage or be in signal communication therewith. In this, the user may access, for example via a website, a program running on the server in order to run or initiate a portion of the method described herein. The medical system 1 may thus comprise a server. It may comprise user interfaces and/or user terminals such as computer, cell phone or tablet, which are set up to communicate with the server.

It is further encompassed by the present disclosure that, other than as shown herein, the medical system 1 is identical to or comprised by the control device or closed-loop control device 150.

FIG. 2 shows the treatment system in a first embodiment with a medical system 1 interacting with a blood treatment apparatus 100 or with its control device or closed-loop control device 150 while the attending physician or medical staff D and a patient P are involved.

The diagnostic device 700, for example the Retinal Vessel Analyzer from the company Imedos, which can operate as an independent device and is already available on the market, is set up or arranged as an independent device, for example in a dialysis station. Patients may be examined therewith or using it, preferably non-invasively. An average examination takes only a few minutes. This may be done in different cycles and does not have to take place before each treatment. An examination at intervals of 2 to 4 weeks seems appropriate.

Thus, in the example of FIG. 2, the diagnostic device 700 determines the values of the ratio of the maximum venous dilatation to the base diameter of the vessel in percent (also: vMax) and the values of the ratio of the maximum arterial dilatation to the base diameter of the vessel in percent aMax (not shown in FIG. 2) and transmits them, for example via a suitable network protocol, to the server that preferably stores the treatment data and patient data. In this, the course of the values of aMax and vMax may also be stored over several examinations or treatment sessions.

In some embodiments, when the respective patient P is logged on to a blood treatment apparatus 100, the blood treatment apparatus 100 sends a request to the server and receives in response the treatment data of the respective patient P. Preferably, a display device 500, which may be part of the blood treatment apparatus 100, may display the measured values for aMax and vMax in a suitable view. Resulting from the measured values, for example, suggested technical parameter values, target treatment parameter values or further treatment modifications may be displayed and suggested or proposed to the attending physician.

After the suggested and determined parameter values or target treatment parameter values have been checked and confirmed by the doctor or by other medical personnel qualified for this purpose, these values may be adopted by the blood treatment apparatus 100 for the upcoming (or ongoing) treatment of the patient P, in particular by the control device or closed-loop control device 150 of the blood treatment apparatus 100; and the blood treatment apparatus 100, in particular its pumps 101, 111, 121, are controlled or regulated individually adapted to the patient P on the basis of the determined parameter values or target treatment parameter values.

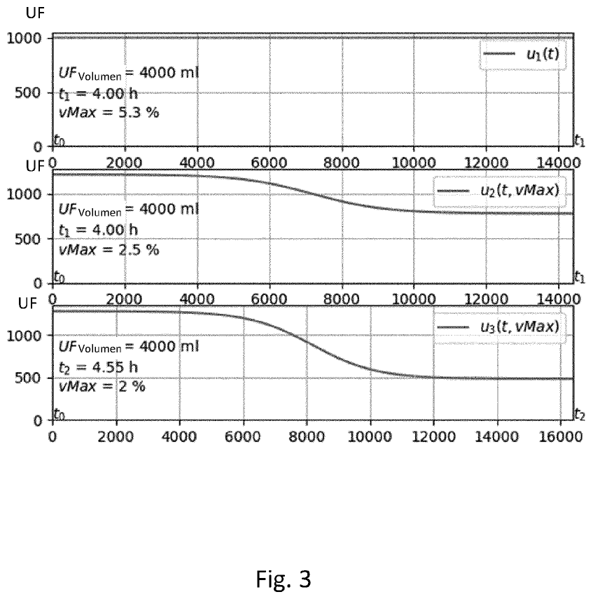

FIG. 3 shows the specification of the ultrafiltration rate, denoted in FIG. 3 as UF (in [ml/h]), over the time duration t₁, t₂, t₃ (in [sec]) as an example of a—here changing over time—technical parameter value.

The values of vMax and aMax as results of the retinal vessel analysis may be classified or subdivided in a variety of ways, and different values for the technical parameters or target treatment parameter values relevant in the treatment of patient P may be assigned to them according to their class or characteristic, and wherein such an assignment may be stored for example in data memories.

A possible subdivision may be carried out as follows, for example in tertiles:

| | |
|---|---|
| lower tertile of vMax | <2.44% |
| middle tertile of vMax | 2.44% bis 4.46% |
| upper tertile of vMax | >4.46% |

and/or

| | |
|---|---|
| lower tertile of aMax | <0.71% |
| middle textile of aMax | 0.71% bis 2.80% |
| upper tertile of aMax | >2.80 % |

These figures are purely exemplary and not to be understood as limiting. These values may serve as standard pre-settings, other values and other subdivisions are also provided.

Depending on vMax, parameter values or target treatment parameter values or changes thereof are suggested. If vMax is below the upper tertile in the present example, a high volume HDF treatment is activated. This is understood here to be a contribution to a cardioprotective dialysis.

Furthermore, in this example, the course of the UF rate is changed over time depending on the results of the retinal vessel analysis. The curve forms shown in FIG. 3 are exemplary. An application to other curve forms is possible; equation (1) should apply to extract the desired ultrafiltration volume U_{g}.

$${U}_{g} = {\int }_{{t}_{0}}^{{t}_{1}} {u}_{1} ( t ) dt = {\int }_{{t}_{0}}^{{t}_{1}} {u}_{2} ( t , vMax ) dt = {\int }_{{t}_{0}}^{{t}_{2}} {u}_{3} ( t , vMax ) dt , \qquad ( 1 )$$

If vMax is within the upper tertile, for example a constant UF rate is applied, see the top curve in FIG. 3.

The ultrafiltration volume U_{g} to be withdrawn is specified, for example, by the attending physician or results from the measurement by a body composition monitor (BCM), if available, etc.

For Uₐ the following results

$${U}_{a} = \frac{{U}_{g}}{{t}_{1} - {t}_{0}} \qquad ( 2 )$$

The following applies

u₁(t)=Uₐ (3)

If vMax is in the middle tertile, the UF rate is thus given here as a function u₂(t,vMax), see the middle curve u₂(t,vMax) in FIG. 3. As a result, more volume is withdrawn per time at the beginning compared with the above-mentioned UF rate of the top representation in FIG. 3 (there: 1000 ml/h) (now: around 1200 ml/h), but the UF rate curve should flatten out again quickly. As a result, the same ultrafiltration volume is initially withdrawn faster than in the uppermost representation, but later more slowly than there.

The following applies by way of example:

$${u}_{2} ( t , vMax ) = \frac{{U}_{0 m} ( vMax ) - {U}_{1} ( vMax )}{1 + {e}^{- k ( - t + {t}_{w} )}} + {U}_{1} ( vMax ) \qquad ( 4 )$$

In this, the following applies:
U_{g} the total ultrafiltration volume withdrawn over the duration of the treatment session
Uₐ is the constant UF rate in the standard case (without modification based on the results of the retinal vessel analysis)
U₀ the standard UF rate at the beginning of the treatment session U₀ₘ(vMax) is the UF rate modified by the results of the retinal vessel analysis at the beginning of treatment
Uₘₐₓ is the maximum allowed UF rate
U₁(vMax) is the UF rate at the end of treatment (modified by the result of retinal vessel analysis)
k is a factor that influences the curvature of the function
t_{w} is the inflection point at which the change of direction of the curvature occurs
t₀ is the time at which the treatment session starts (at t=0)
t₁ is the time at which the treatment session ends
t₂(vMax) is the time, being modified based on the results of the retinal vessel analysis, at which the treatment session ends

If vMax is in the lower tertile, the middle curve u₂(t,vMax) of FIG. 3 can be exemplarily modified to u₃(t,vMax). For this purpose, in addition to the modifications of u₂(t,vMax), the duration of the treatment session (dialysis time) is extended, as shown by the lowest curve u₃(t,vMax) of FIG. 3. FIG. 3 thus shows different UF rates over time, as well as the modification of the treatment duration, which for u₃ in FIG. 3 was increased from 4 hours (4 h) to e.g. 4 hours and 33 minutes (4.55 h).

All parameters can be modified as needed, e.g., by the attending physician.

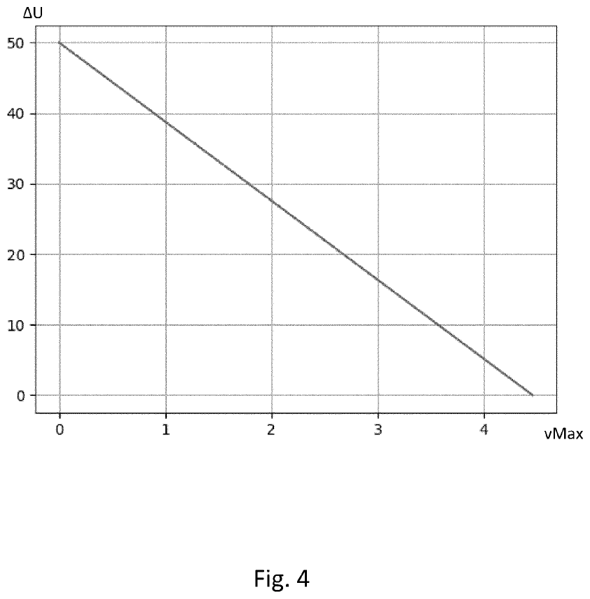

FIG. 4 shows an exemplary procedure when proposing a change ΔU [in %] from an existing pre-set specification U₀ as a specification for the technical parameter value of the UF rate depending on the venous, maximum dilatation vMax [in %].

The parameter U₀, which is understood here as the standard value or pre-set specification for the UF rate, is influenced or changed by the results from the retinal vessel analysis plotted along the x-axis, for example as follows:

Based on the measured value of vMax, U₀, a constant starting value or standard value of, for example, 1000 ml/h, is increased by, for example, a maximum of 50% of U₀.

This is done, for example by interpolation using a straight line, as shown in FIG. 4.

U₀ₘ(vMax)=U₀*(1+ΔU(vMax)) (5)

This curve course and the values used are purely exemplary and are not to be understood as limiting. Any modifications are possible.

The following should preferably apply:

ΔU(vMax)≥0 and U₀ₘ(VMax)≤Uₘₐₓ

To keep the withdrawn UF volume unchanged, U₁(vMax)) can be calculated as follows by inserting (3), (4) and (5) into (1) and solving it for U₁(vMax):

$${U}_{1} ( vMax ) = \frac{kt ( {U}_{a} - {U}_{0 m} ( vMax ) )}{{log}_{e} ( {e}^{k ( t - {t}_{w} )} + 1 )} + {U}_{0 m} ( vMax ) for t = {t}_{1} \qquad ( 6 )$$

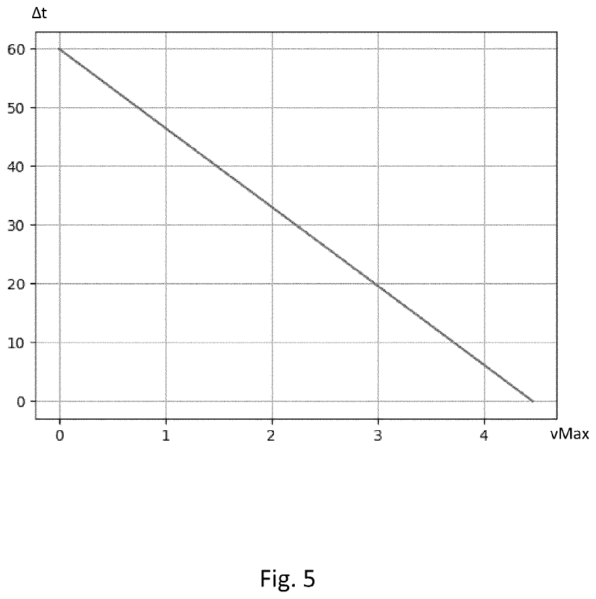

FIG. 5 shows the dependence of the change Δt (in [sec]) in the duration of the treatment session (as an example of a target treatment parameter) on the venous maximal dilatation vMax (in [%]).

If vMax is in the lower tertile, then, as stated above, the middle curve u₂(t,vMax) of FIG. 3 can be changed exemplarily to u₃(t,vMax). For this purpose, in addition to the modifications of u₂(t,vMax), the duration of the treatment session (dialysis time) is extended (see also the lowest curve u₃(t,vMax) of FIG. 3). With an unfavorable calcium/phosphate balance, calcification leads to increased stenoses in the vascular system. Prolonged dialysis time may improve the reduction or breaking down of excess phosphate, which may be an advantage in the treatment of some patients that can be identified by the present systems, methods, and devices.

For this purpose, as with u₂(t,vMax), see above or the middle curve in FIG. 3, the change ΔU for modifying the pre-set specification U₀ for the UF rate is first determined in dependance of the venous maximum dilatation vMax at the beginning of the treatment session or the dialysis.

Furthermore, a modified end time or duration of the treatment session or the dialysis time is calculated as a change Δt to modify the pre-set specification t₁ for duration as an example of a target treatment parameter. This is done, for example, by interpolation using a straight line as shown in FIG. 5.

t₂(vMax)=t₁+Δt(vMax) (7)

This curve progression and the values used are purely exemplary and are not to be understood as limiting. Any modifications are possible.

The following should apply:

Δt(vMax)≥0

There are hardly any restrictions on extending the duration of dialysis. The longer dialysis lasts, the milder it is for the patient. A reduction in blood flow seems appropriate here, which may lead to a further change in the specification of a technical parameter value (in this case, the preset blood pump rate). In practice, however, dialysis duration may also be limited by economic and patient-related factors.

To ensure that the total withdrawn ultrafiltration volume remains unchanged, U₁(vMax) may be calculated by inserting (3), (4) and (5) in (1) and solving it for U₁(vMax):

$${U}_{1} ( vMax ) = \frac{kt ( {U}_{a} - {U}_{0 m} ( vMax ) )}{{log}_{e} ( {e}^{k ( t - {t}_{w} )} + 1 )} + {U}_{0 m} ( vMax ) \qquad ( 8 )$$

$$for t = {t}_{2} ( vMax )$$

If the dialysis machine has the option of sodium management, then, when vMax is below the upper tertile, sodium management is further activated with a 0 balance, which may correspond to a possible value of the sodium balance or of the target sodium level as a possible target treatment parameter.

The attending physician may respectively modify the target sodium level in in order to achieve a positive effect on the intravascular volume. If the sodium balance is zero, the patient's sodium level is the same before and after treatment.

In addition, if vMax is below the upper tertile, then for example the use of the Body Composition Monitor (in short: BCM, which may optionally also be part of the treatment system) is recommended. If the BCM option is switched on, the treatment system or the treatment apparatus may request a new measured value at regular intervals, e.g., every 4 weeks, if such a value will not be, or has not been, transmitted automatically, e.g., by SmartCard or the like.

The determined values of vMax and aMax may be displayed as a graphic in some embodiments. Thus, a long-term history may be presented to the attending physician. In the case of increasing values of vMax and aMax, dialysis treatment that is as cardioprotective as possible may be indicated.

In some embodiments, some or all of the changes to the parameter values or target treatment parameter values are given as suggestions to the attending personnel. In certain embodiments, these suggestions must be explicitly accepted before the changes are activated or made active, i.e., transmitted to the control device or closed-loop control device 150 of the blood treatment apparatus 100 (see FIG. 1 and FIG. 2).

The disclosure of the figures and the explanation thereof are only exemplary. In particular, the curves shown do not have to be linear. The respective curve progression, as well as the absolute values, could be determined differently or modified by the attending physician.

### LIST OF REFERENCE NUMERALS

1 medical system
5 calculation device
9 source for citrate solution, here exemplarily
10 as a citrate bag
12 calcium pump
13 source for calcium solution, calcium bag
15 citrate pump
25 addition site for Heparin (optional)
29 venous blood chamber (optional)
31 de-aeration device
51 input interface
53 output interface
100 blood treatment apparatus
101 blood pump
102 dialysate outlet line
104 dialysis liquid inlet line
111 pump for substitute fluid
121 pump for dialysis liquid, dialysis liquid pump
131 pump for dialysate or effluent in effluent inlet line; pump for ultrafiltration, ultrafiltration pump
150 control device or closed-loop control device
200 source with dialysis liquid
201 source with substitute fluid, optional
300 extracorporeal blood circuit
301 first line (arterial line section)
302 (first) tube clamp
303 blood filter or dialyzer
303a dialysis liquid chamber
303b blood chamber
303c semipermeable membrane
305 second line (venous line section)
306 (second) tube clamp
400 effluent bag
500 display device
700 diagnostic device
D attending doctor, medical staff
H1 bag heater
H2 bag heater
P patient
PS1 arterial sensor
PS2 pressure sensor (optional, pre-hemofilter)
PS3 pressure sensor
PS4 pressure sensor
t₁, t₂, t₃ time duration [sec]
Δt change in time duration
UF ultrafiltration rate [ml/h]
ΔU change in the ultrafiltration rate
aMax arterial dilatation; ratio of maximum arterial dilatation to the base diameter of the vessel as a percentage
vMax venous dilatation ration of maximum venous dilatation to base diameter of the vessel as a percentage

1-18. (canceled)

19. A medical system for specifying adjustable values of a blood treatment apparatus, the medical system comprising:
a calculation device with an input interface for an input and/or for reading in results respectively determined by a diagnostic device for performing a retinal vessel analysis of a patient;
an output interface for outputting at least one technical parameter value for treatment of the patient and/or at least one target treatment parameter value for treatment of the patient by the blood treatment apparatus, and/or for outputting suggested changes to existing pre-set specifications or pre-settings for the at least one technical parameter value and/or the at least one target treatment parameter value;
wherein the calculation device is programmed to:
determine, based on results being entered or read in via the input interface, the at least one target treatment parameter value or the at least one technical parameter value, and/or a suggested or advised change from the pre-set specifications, for controlling or regulating the blood treatment apparatus for the treatment of the patient; and
output the at least one technical parameter value and/or the at least one target treatment parameter value, or a change in the at least one technical parameter value and/or the at least one target treatment parameter value, via the output interface.

20. The medical system according to claim 19, wherein the results are, build upon, or encompass venous and/or arterial dilatation.

21. The medical system according to claim 19, further comprising a display device configured or programmed to display the results and/or the at least one technical parameter value and/or the at least one target treatment parameter value, or a change in the at least one technical parameter value and/or the at least one target treatment parameter value.

22. The medical system according to claim 19, wherein the output interface comprises a visual or graphical interface for the user or is connected to a graphical display device.

23. The medical system according to claim 19, wherein the output interface comprises an interface with a control device or closed-loop control device of the blood treatment apparatus.

24. The medical system according to claim 19, wherein the output interface comprises an interface with a server, a network protocol, and/or a data storage.

25. The medical system according to claim 19, wherein the medical system comprises a mobile device, handheld device, cell phone, smartphone, tablet, and/or an application suitable therefor.

26. The medical system according to claim 19, further comprising a diagnostic device for executing a retinal vessel analysis of the patient.

27. The medical system according to claim 19, wherein the at least one technical parameter value or at least one target treatment parameter value determined by the calculation device relate to, or comprise, an ultrafiltration volume for the current or the upcoming treatment session, a treatment duration thereof, a treatment type, a sodium management and/or liquid management, or changes thereof.

28. The medical system according to claim 19, wherein the medical system is a control device or closed-loop control device of the blood treatment apparatus or part thereof, encompasses the control device or closed-loop control device, or is in signal communication therewith.

29. The medical system according to claim 28, wherein the control device or closed-loop control device is programmed to control or regulate the blood treatment apparatus, which comprises a conveying device for a liquid on the basis of the calculated at least one technical parameter value or the at least one target treatment parameter value.

30. The medical system according to claim 29, wherein the conveying device comprises an ultrafiltration pump, a blood pump and/or a dialysis liquid pump.

31. A blood treatment apparatus, embodied as a dialysis apparatus, comprising, or connected to, the medical system according to claim 19.

32. The blood treatment apparatus according to claim 31, embodied as a hemodialysis apparatus, hemofiltration apparatus or hemodiafiltration apparatus, an apparatus for acute renal replacement therapy, for chronic renal replacement therapy, or for continuous renal replacement therapy (CRRT).

33. A treatment system comprising:
one or several blood treatment apparatuses, each embodied as a dialysis apparatus; and
a medical system according to claim 19;
wherein at least one or more of the blood treatment apparatuses and the medical system are present separately from each other.

34. A method for preparing for an upcoming treatment or treatment session of a patient, which is to be carried out by using a blood treatment apparatus, said method comprising:
providing a medical system according to claim 19;
entering at least one result determined by the diagnostic device into the input interface of the calculation device;
reading, from the output interface, the at least one technical parameter value and/or the at least one target treatment parameter value, or at least one change of the at least one technical parameter value and/or the at least one target treatment parameter value, for a treatment of the patient using the blood treatment apparatus; and
entering the at least one technical parameter value and/or the at least one target treatment parameter value, or the at least one change of the at least one technical parameter value and/or the at least one target treatment parameter value, as a set value into an input interface of the blood treatment apparatus.

35. A digital storage medium, with electronically readable control signals, designed to interact with a programmable computer system such that a conventional calculation device is reprogrammed into a calculation device of a medical system according to claim 19.

36. The digital storage medium of claim 35, in the form of a floppy disk, CD or DVD, EPROM, FRAM or SSD.

37. A computer program product, as a signal wave or having a program code stored on a machine-readable carrier, for interacting with a programmable computer system such that a conventional calculation device is reprogrammed into a calculation device of a medical system according to claim 19.

38. A computer program with a program code to reprogram a conventional calculation device into a calculation device of a medical system according to claim 19, when the computer program runs on a computer.
